(19) United States
(12) Patent Application Publication
(10) Pub. No.: US 2010/0223953 A1
(43) Pub. Date: Sep. 9, 2010

(54) DEVICE FOR HOLDING FRAGRANCE

(75) Inventor: Chih-Chen Juan, Taichung County (TW)

Correspondence Address: APEX JURIS, PLLC, 12733 LAKE CITY WAY NORTHEAST, SEATTLE, WA, 98125, US

(73) Assignee: YONGMART MANUFACTURING CO., LTD., Taichung County (TW)

(21) Appl. No.: 12/715,935

(22) Filed: Mar. 2, 2010

(30) Foreign Application Priority Data
Mar. 4, 2009 (TW) 098203279

(51) Int. Cl. A44C 15/00; A61L 9/04; A44C 9/00

(52) U.S. Cl. 63/1.15; 239/36

(57) ABSTRACT

A device for holding fragrance having a container with a porous sidewall and an absorber received in the container. The absorber absorbs perfume to produce fragrance. The device of the present invention may be a ring, earring, necklace, brooch, hair adornment, pin, bracelet etc. Moreover, the device of the present invention may be a perfume spray used in the car or bathroom also.

### BACKGROUND OF THE INVENTION

1. Field of the Invention

The present invention relates generally to perfume, and more particularly to a device for holding fragrance.

2. Description of the Related Art

Nowadays, more and more people wear perfume in formal occasions. For both women and men, in some occasions wearing perfume is considered as necessary and proper social manners.

However, some people are allergic to the touch of perfume, and wearing perfume will provoke their allergy. Such people cannot wear perfume. For some occasions, it is troublesome.

There are some natural perfumes which will not irritate skin. However, such perfumes are few and expensive, and only few fragrances are available for this purpose, which cannot satisfy those consumers.

### SUMMARY OF THE INVENTION

The primary objective of the present invention is to provide a device for holding fragrance, which may be worn as an accessory and produce fragrance without contacting skin.

According to the objective of the present invention, a device for holding fragrance having a container with a porous sidewall and an absorber received in the container. The absorber absorbs perfume, holds it and produces fragrance.

### BRIEF DESCRIPTION OF THE DRAWINGS

FIG. 1 is a perspective view of a first preferred embodiment of the present invention;

FIG. 2 is a perspective view of a second preferred embodiment of the present invention; and

FIG. 3 is a perspective view of a third preferred embodiment of the present invention.

### DETAILED DESCRIPTION OF THE INVENTION

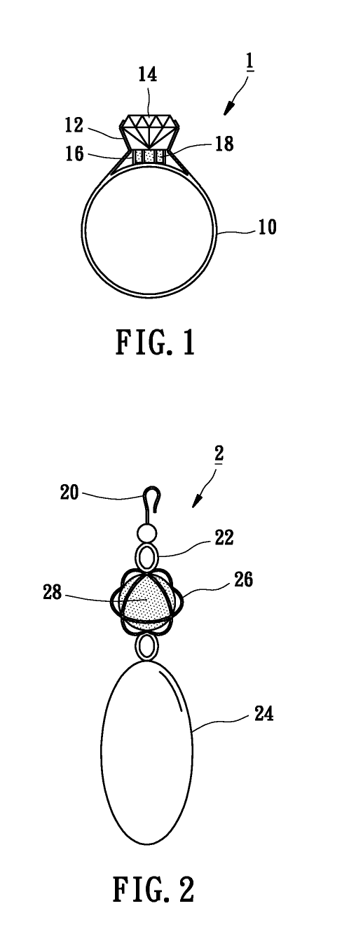

As shown in FIG. 1, the first embodiment of the present invention provides a ring 1, which includes a ring member 10, a base 12, and an ornament 14. The ring 1 further includes a container 16, which has a porous sidewall, between the ring member 10 and the base 12 and an absorber 18 received in the container 16. The container 16 is constrained by several wires to form a cage. The absorber 18 is made of a macromolecule material that may hold fragrance for a long time.

One may drop perfume into the absorber 18 to let perfume be sucked into it that the absorber 18 may produce fragrance for a long time. The container 16 may separate the absorber 18 from skin, and the porous sidewall of the container 16 may pass fragrance that one drops into the absorber 18 without having to worry about the allergy problem. The ring 1 of the present invention is an accessory itself and may also produce fragrance from the absorber 18.

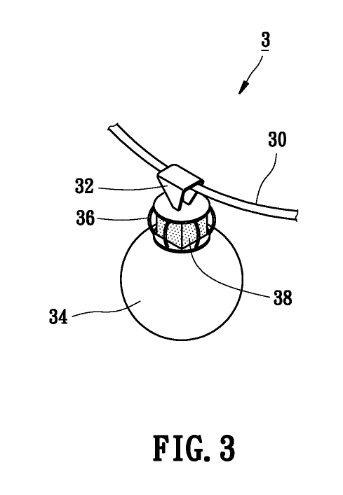

With the idea of the present invention, this device may be incorporated in other objects, such as an earring 2 as shown in FIG. 2. The earring 2 has a fastener 20, which is a hook, a chain 22, and an ornament 24. A porous container 26 is provided on the chain 22, and an absorber 28 is received in the container 26. The container 26 is constrained by three metallic rings to form a ball-like cage, and the absorber 28 is a ball. FIG. 3 shows a necklace 3 having a chain 30, a connector 32 on the chain 30, and an ornament 34 connected to the connector 32. Same as above, a porous container 36 is provided between the chain 30 and the ornament 34, and an absorber 38 is received in the container 36.

The present invention may be incorporated in various accessories, such as brooch, hair adornment, pin, bracelet etc. Moreover, the present invention may as well be incorporated as perfume spray used in the car or bathroom.

What is claimed is:

1. A device for holding fragrance, comprising a container with a porous sidewall and an absorber received in the container, wherein the absorber absorbs perfume.

2. The device as defined in claim 1, wherein the container is constrained by a plurality of wires to form a cage.

3. The device as defined in claim 1, wherein container is constrained by a plurality of rings to form a ball-like cage.

4. The device as defined in claim 1, wherein the absorber is made of a macromolecule material.

5. The device as defined in claim 1, wherein the device is a ring having a ring member and an ornament, and the container is provided between the ring member and the ornament.

6. The device as defined in claim 1, wherein the device is an earring having a fastener and an ornament, and the container is provided between the fastener and the ornament.

7. The device as defined in claim 1, wherein the device is a bracelet having a chain, a connector and an ornament, and the container is provided on the connector.

8. The device as defined in claim 1, wherein the device is worn on human body.

9. The device as defined in claim 1, wherein the device is a perfume spray.
